(19) United States
(12) Patent Application Publication
(10) Pub. No.: US 2024/0041733 A1
(43) Pub. Date: Feb. 8, 2024

(54) DENTAL CARE COMPOSITION AND METHOD OF PROMOTING CALCIFICATION IN LIVING BODY TISSUE

(71) Applicants: KINJIRUSHI CO., LTD., Nagoya-shi (JP); TOKYO DENTAL COLLEGE, Tokyo (JP)

(72) Inventors: Isao Okunishi, Nagoya-shi (JP); Yoshiyuki Shibukawa, Tokyo (JP); Maki Kimura, Tokyo (JP)

(21) Appl. No.: 18/268,439

(22) PCT Filed: Dec. 22, 2021
(86) PCT No.: PCT/JP2021/047722

§ 371 Date: Jun. 20, 2023

(30) Foreign Application Priority Data
Dec. 22, 2020 (JP) 2020-212421

(51) Int. Cl. A61K 8/46; A61K 8/02; A61Q 11/00

(52) U.S. Cl. CPC A61K 8/46; A61K 8/0216; A61Q 11/00; A61K 2800/92

(57) ABSTRACT

A dental care composition including an isothiocyanate having a sulfinyl group (—S(═O)—), a sulphonyl group (—S(═O)₂—), a sulfide bond (—S—), or an aryl group, and an isothiocyanate group (—N═C═S). And, a composition used to promote activation of a TRPA1 channel and to promote activation of a reaction catalyzed by carbonic anhydrase.

### CROSS-REFERENCE TO RELATED APPLICATION

This international application claims the benefit of Japanese Patent Application No. 2020-212421 filed on Dec. 22, 2020 with the Japan Patent Office, and the entire disclosure of Japanese Patent Application No. 2020-212421 is incorporated herein by reference.

### TECHNICAL FIELD

The present disclosure relates to a dental care composition, and a method of promoting calcification in a living body tissue (for example, a tooth and a periodontal tissue).

### BACKGROUND ART

For example, hard tissues such as teeth and bones are known as living body tissues that calcify. The dentin of a tooth is a tissue surrounding the dental pulp which is commonly known as a nerve of a tooth. The dentin is covered with the enamel which is the most superficial layer of a tooth. In the dentin, a myriad of dentinal tubule radially runs from the border of the dentin and the enamel to the dental pulp. Processes of odontoblasts protrude in the dentinal tubule; the odontoblasts, which exist at the outermost layer of the dental pulp, form and repair the dentin.

The inside of the dentinal tubule is filled with tissue fluid; and nutrients are carried to entire teeth from blood vessels in the dental pulp through the dentinal tubule.

The dentinal tubule also serves as a mechanism to transmit an external stimulus applied to a tooth, such as a temperature stimulus, a mechanical stimulus, and a chemical stimulus, to the dental pulp. These external stimuli are strongly transmitted to the dental pulp when the dentin is exposed because of the enamel being scaled off due to abrasion, dental caries, or the like, and when the dentin, originally covered by the gum, is exposed because of regression of the gum due to periodontitis, aging, or the like. It consequently causes a symptom of hyperesthesia.

When an external stimulus is transmitted to the dental pulp, various biological reactions to protect the teeth occur. For example, in an area where the dentin is eroded, a dentin called a tertiary dentin is newly formed so as to protrude towards the dental pulp. The tertiary dentin is considered to be formed by calcification of an organic substrate. The tertiary dentin is known to be further categorized into a reactionary dentine and a reparative dentine depending on the strength of the external stimulus. On the surface of the exposed dentin, the dentinal tubule is closed by calcification that occurs inside the dentinal tubule. These phenomenon of calcification is considered to be caused by the odontoblasts.

In recent years, it has been proven that various membrane channel responsive to external stimuli expressed in the odontoblasts are involved in calcification of the dentin. For example, Non-Patent Document 1 discloses that calcification of the dentin is inhibited by an addition of an inhibitor of TRPA1 (TRP: transient receptor potential) channel expressed in the odontoblasts.

### PRIOR ART DOCUMENTS

### Non-Patent Document

Non-Patent Document 1: Maki Kimura and 14 other authors, “Activation of an alkaline-sensitive TRPA1 channel in odontoblasts promotes calcification”, The Journal of the Tokyo Dental College Society, Tokyo Dental College Academic Society, 2016, Vol. 116, No. 3, p. 231

### SUMMARY OF THE INVENTION

### Problems to be Solved by the Invention

As a result of an earnest examination focusing on chemical compounds that activate the TRPA1 channel, the inventors of the invention of the present application have newly found that a specific chemical compound promotes calcification in a living body tissue.

One aspect of the present disclosure preferably provides a technique to promote calcification in a living body tissue (for example, in a tooth and a periodontal tissue).

### Means for Solving the Problems

One mode of the present disclosure is a dental care composition including an isothiocyanate having a sulfinyl group (—S(═O)—), a sulphonyl group (—S(═O)₂—), a sulfide bond (—S—), or an aryl group, and an isothiocyanate group (—N═C═S).

In one mode of the present disclosure, the isothiocyanate may include a methylsulfinylalkyl isothiocyanate.

In one mode of the present disclosure, the isothiocyanate may include a 6-methylsulfinylhexyl isothiocyanate.

In one mode of the present disclosure, the dental care composition may be a chewing gum. If the dental care composition has a recommended intake per use, the content of the isothiocyanate in the recommended intake per use may be greater than or equal to 0.036 μmol. If the dental care composition does not have a recommended intake per use, the content of the isothiocyanate in one piece of the dental care composition may be greater than or equal to 0.036 mol.

In one mode of the present disclosure, the dental care composition may be a candy. If the dental care composition has a recommended intake per use, the content of the isothiocyanate in the recommended intake per use may be greater than or equal to 0.12 μmol. If the dental care composition does not have a recommended intake per use, the content of the isothiocyanate in one piece of the dental care composition may be greater than or equal to 0.12 μmol.

In one mode of the present disclosure, the dental care composition may be a tablet-shaped sweet. If the dental care composition has a recommended intake per use, the content of the isothiocyanate in the recommended intake per use may be greater than or equal to 0.012 μmol. If the dental care composition does not have a recommended intake per use, the content of the isothiocyanate in one piece of the dental care composition may be greater than or equal to 0.012 μmol.

In one mode of the present disclosure, the dental care composition may be a lozenge. If the dental care composition has a recommended intake per use, the content of the isothiocyanate in the recommended intake per use may be greater than or equal to 0.084 μmol. If the dental care composition does not have a recommended intake per use, the content of the isothiocyanate in one piece of the dental care composition may be greater than or equal to 0.084 mol.

In one mode of the present disclosure, the dental care composition may be a dentifrice. If the dental care composition has a recommended amount per use, the content of the isothiocyanate in the recommended amount per use may be greater than or equal to 0.036 μmol. If the dental care composition does not have a recommended amount per use, the content of the isothiocyanate in 1 g of the dental care composition may be greater than or equal to 0.036 μmol.

In one mode of the present disclosure, the dental care composition may be used for one or more purposes selected from improvement of hyperesthesia and prevention of hyperesthesia.

In one mode of the present disclosure, the dental care composition may be used to strengthen dental tissues.

In one mode of the present disclosure, the dental care composition may be used to promote calcification of dentin.

Another mode of the present disclosure is a method of promoting calcification of the dentin (excluding medical practices to a human). The method includes applying a dental care composition to a tooth; the dental care composition includes isothiocyanate having a sulfinyl group (—S(═O)—), a sulphonyl group (—S(═O)₂—), a sulfide bond (—S—), or an aryl group, and an isothiocyanate group (—N═C═S).

In one mode of the present disclosure, the isothiocyanate may include a methylsulfinylalkyl isothiocyanate.

In one mode of the present disclosure, the isothiocyanate may include a 6-methylsulfinylhexyl isothiocyanate.

In one mode of the present disclosure, the dental care composition may be a composition, at least a part of which dissolves in saliva in an oral cavity. The method may include applying the dental care composition into an oral cavity such that the concentration of the isothiocyanate in the saliva is greater than or equal to 0.04 mol/m³.

Another mode of the present disclosure is a method of promoting calcification of dentin (excluding medical practices to a human). The method includes having a liquid containing an isothiocyanate come into contact with a tooth; the isothiocyanate have a sulfinyl group (—S(═O)—), a sulphonyl group (—S(═O)₂—), a sulfide bond (—S—), or a phenyl group, and an isothiocyanate group (—N═C═S). The concentration of the isothiocyanate in the liquid is greater than or equal to 0.04 mol/m³.

Another mode of the present disclosure is a composition used to promote activation of a TRPA1 channel and to promote activation of a reaction catalyzed by carbonic anhydrase.

In one mode of the present disclosure, the aforementioned composition may include an isothiocyanate (excluding allyl isothiocyanate and n-alkyl isothiocyanate) as an active component.

In one mode of the present disclosure, the aforementioned isothiocyanate may be at least one of a 6-methylsulfinylhexyl isothiocyanate, a 6-methylthiohexyl isothiocyanate, or a 6-methylsulfonylhexyl isothiocyanate.

### Effects of the Invention

One mode of the present disclosure provides a technique that enables promotion of calcification of a living body tissue.

### BRIEF DESCRIPTION OF THE DRAWINGS

FIG. 1 shows evaluation results on calcification promoting action of a 6-methylsulfinylhexyl isothiocyanate at amounts of 0.04 nM to 400 nM.

FIG. 2 shows evaluation results on calcification promoting action of a 6-methylsulfinylhexyl isothiocyanate at amounts of 4 μM to 400 μM.

FIG. 3 shows evaluation results on calcification promoting action of a 6-methylthiohexyl isothiocyanate.

FIG. 4 shows evaluation results on calcification promoting action of a 6-methylsulfonylhexyl isothiocyanate.

FIG. 5 shows evaluation results on calcification promoting action of a 4-methylsulfinylbutyl isothiocyanate.

FIG. 6 shows evaluation results on calcification promoting action of an 8-methylsulfinyloctyl isothiocyanate.

FIG. 7 shows evaluation results on calcification promoting action of a phenethyl isothiocyanate.

FIG. 8 shows evaluation results on calcification promoting action of a 4-methylsulfinylbutyl amine.

FIG. 9 shows evaluation results on calcification promoting action of an n-hexyl isothiocyanate.

FIG. 10 shows evaluation results on calcification promoting action of an allyl isothiocyanate at amounts of 1 μM to 100 μM.

FIG. 11 shows evaluation results on calcification promoting action of the allyl isothiocyanate at an amount of 500 μM.

FIG. 12 shows evaluation results on calcification inhibition tests when dorzolamide, an inhibitor of carbonic anhydrase, is added.

FIG. 13 shows a result on an analysis of a reactionary dentine forming ability of a 6-methylsulfinylhexyl isothiocyanate (6-MSITC) using a rat. FIG. 13 shows sliced images (CT images) of samples of both mandibles of the rat scanned by micro CT two weeks after the rat was administrated with saline solution or the 6-MSITC, and also enlarged images of the rat's teeth on the CT images. The image on the left shows a result of a group administrated with saline solution (control; A1); the image on the right shows a result of a group administrated with 500 μM of the 6-MSITC (A2). In FIG. 13, dentin is D, enamel is E, dental pulp is P, and cavity is C. Thick arrows and narrow arrows in the image are pointing areas where formations of new reactionary dentins are observed. The thick arrows are pointing high calcification areas where the extent of calcification is large. The narrow arrows are pointing low calcification areas where the extent of calcification is small.

FIG. 14 shows a result on an analysis of a reactionary dentine forming ability of the 6-MSITC using a rat. FIG. 14 shows a three-dimensional construction image produced by three-dimensional stereoscopic construction using sliced data which was taken when the sample of both mandibles of the rat described in FIG. 13 was scanned by micro CT. The image on the left shows a result of a group administrated with saline solution (control; A1); the image on the right shows a result of a group administered with 500 μM of 6-MSITC (A2). In FIG. 14, dentin is D, enamel is E, dental pulp is P, and cavity is C. Thick arrows are pointing areas where formations of new reactionary dentins are observed (high calcification area).

### MODE FOR CARRYING OUT THE INVENTION

[Composition Promoting Calcification in Living Body Tissue]

A composition that promotes calcification in a living body tissue according to the present disclosure can be provided in any one of the modes given below.

A dental care composition according to one mode of the present disclosure includes an isothiocyanate having a sulfinyl group (—S(═O)—), a sulphonyl group (—S(═O)₂—), a sulfide bond (—S—), or an aryl group, and an isothiocyanate group (—N═C═S).

According to the study by the inventors of the present disclosure, an isothiocyanate having such a structure is able to promote formation, regeneration, and calcification of the dentin of the teeth. “Calcification of the dentin” described in the present disclosure includes various calcification phenomena that occur in the dentin of the teeth. Examples of the calcification that occurs in the dentin include, as mentioned above, calcification involved in the formation of the reactionary dentine and a reparative dentine, as well as calcification inside the dentinal tubules. Hereinafter, an action to promote calcification of the dentin is also simply called a “calcification promoting action”.

Another mode of the present disclosure is a composition used to promote activation of a TRPA1 channel and to promote activation of a reaction catalyzed by carbonic anhydrase. “The composition used to promote activation of TRPA1 channel and to promote activation of a reaction catalyzed by the carbonic anhydrase (hereinafter also referred to as “TRPA1 and CA activation promoting composition)” is a composition that promotes activation of TRPA1 channel and activation of a reaction catalyzed by the carbonic anhydrase by application of the composition to the living body tissue. More specifically, the TRPA1 and CA activation promoting composition is a composition that promotes activation of TRPA1 channel and activation of a reaction catalyzed by the carbonic anhydrase in cells that form a living body tissue by having the composition come into contact with the living body tissue. Examples of the living body tissue include an oral tissue, and a periodontal tissue. The living body tissue may be, for example, a dental tissue or a periodontal tissue. The living body tissue may be, for example, a tooth or an alveolar bone. In the present disclosure, a hard tissue such as a bone or a tooth is assumed as the living body tissue; calcification of a soft tissue such as a blood vessel (that is, ectopic calcification) is not assumed. If the living body tissue is a tooth, the TRPA1 and CA activation promoting composition may be a composition that promotes activation of the TRPA1 channel and activation of a reaction catalyzed by the carbonic anhydrase in an odontoblast by having the composition come into contact with the tooth. The composition may also be a composition that promotes activation of a receptor of the composition including at least the TRPA1 channel.

Promotion of activation of the TRPA1 channel in a cell by an application of the TRPA1 and CA activation promoting composition can be examined, for example, by confirming (observing) an increase in the activity of the TRPA1 channel in a case where the composition is in contact with a cell that expresses the TRPA1 compared with a case where the composition is not in contact with a cell that expresses the TRPA1. The activity of the TRPA1 channel can be measured by methods well-known by a person skilled in the art. For example, a potential fixing patch clamp, a current fixing patch clamp, and any other patch clamp modes can be used as a method to measure the activity of the TRPA1. As a method for measuring the activity of the TRPA1, for example, intracellular calcium imaging using various fluorogenic reagents, intracellular ion imaging by forced expression of fluorescent protein using fluorescence resonance energy transfer (FRET), intracellular signal imaging using various fluorogenic reagent or FRET, fluorescence analysis by a spectrophotofluorometer or a flow cytometer using various fluorogenic reagent or FRET, and a molecular biological method (such as PCR and protein expression analysis) can be used.

Activation of a reaction catalyzed by the carbonic anhydrase in a cell by an application of the TRPA1 and CA activation promoting composition can be examined, for example, by confirming (observing) an increase in the activity of the carbonic anhydrase in a case where the composition is in contact with a cell that expresses the carbonic anhydrase compared with a case where the composition is not in contact with the cell that expresses the carbonic anhydrase. The activity of the carbonic anhydrase can be measured by methods well-known by a person skilled in the art. For example, the methods same as those that measure the activity of the TRPA1 can be used as methods for measuring the activity of the carbonic anhydrase.

According to the study by the inventors of the present disclosure, the TRPA1 and CA activation promoting composition can promote calcification of the living body tissue by promoting the activation of the TRPA1 channel and the activation of a reaction catalyzed by the carbonic anhydrase in cells that form the living body tissue. If the living body tissue is a tooth, for example, the TRPA1 and CA activation promoting composition can promote calcification of the tooth (specifically, the dentin) by promoting the activation of the TRPA1 channel and the activation of a reaction catalyzed by the carbonic anhydrase in the odontoblast by having the composition come into contact with the tooth.

The TRPA1 and CA activation promoting composition may include, for example, an isothiocyanate (excluding an allyl isothiocyanate and an n-alkyl isothiocyanate). In the present disclosure, the n-alkyl isothiocyanate means an alkyl isothiocyanate having no substituents at its terminals. In other words, the n-alkyl isothiocyanate is an isothiocyanate (R—N═C═S; where R is a substituent) whose R is an alkyl group having no substituent at the terminal (specifically, the terminal opposite to —N═C═S group). The isothiocyanate (excluding the allyl isothiocyanate and the n-alkyl isothiocyanate) may be, for example, an isothiocyanate having a sulfinyl group (—S(═O)—), a sulphonyl group (—S(═O)₂—), a sulfide bond (—S—), or an aryl group, and an isothiocyanate group (—N═C═S).

[Isothiocyanate]

The isothiocyanate may be, for example, an alkyl isothiocyanate having a methyl sulfinyl group, a methyl sulphonyl group, a methylthio group, or an aryl group at its terminal. Examples of such isothiocyanate include a methylsulfinylalkyl isothiocyanate, a methylsulphonylalkyl isothiocyanate, a methylthioalkyl isothiocyanate, and an arylalkyl isothiocyanate. A phenyl group is particularly desirable as an aryl group.

Examples of a methylsulfinylalkyl isothiocyanate include a 3-methylsulfinylpropyl isothiocyanate, a 4-methylsulfinylbutyl isothiocyanate, a 5-methylsulfinylpentyl isothiocyanate, a 6-methylsulfinylhexyl isothiocyanate, a 7-methylsulfinylheptyl isothiocyanate, and an 8-methylsulfinyloctyl isothiocyanate.

Examples of a methylsulphonylalkyl isothiocyanate include a 6-methylsulfonylhexyl isothiocyanate.

Examples of a methylthioalkyl isothiocyanate include a 3-methylthiopropyl isothiocyanate, a 4-methylthiobutyl isothiocyanate, a 5-methylthiopentyl isothiocyanate, a 6-methylthiohexyl isothiocyanate, 7-methylthioheptyl isothiocyanate, and an 8-methylthiooctyl isothiocyanate.

Examples of an arylalkyl isothiocyanate include a benzil isothiocyanate, and a phenethyl isothiocyanate.

These isothiocyanates may be used alone or in combination of two or more isothiocyanates.

Due to a high calcification promoting action, it is preferable to include at least one isothiocyanate selected from a group consisting of a 4-methylsulfinylbutyl isothiocyanate, a 6-methylsulfinylhexyl isothiocyanate, a 6-methylsulfonylhexyl isothiocyanate, a 6-methylthiohexyl isothiocyanate, and a phenethyl isothiocyanate. It is more preferable to include a 6-methylthiohexyl isothiocyanate as the isothiocyanate.

In terms of being able to obtain a high calcification promoting action, it is preferable that, as an isothiocyanate, the carbon number (excluding the carbon number of the substituent at the terminal) of the alkyl group having a substituent at the terminal is 3 to 16, more preferably 3 to 8, and yet more preferably 4 to 8, and the most preferably 6.

The substituent of the isothiocyanate is preferably a sulfur-containing substituent or an aryl group. The sulfur-containing substituent is preferably a sulfinyl group (—S(═O)—), a sulphonyl group (—S(═O)₂—), or a sulfide bond (—S—), more preferably a sulfinyl group (—S(═O)—), a sulphonyl group (—S(═O)₂—) or a sulfide bond (—S—), yet more preferably a sulfinyl group (—S(═O)—) or a sulphonyl group (—S(═O)₂—) and the most preferably a sulfinyl group (—S(═O)—). The aryl group has one or more substituents combined, or no substituents combined; and the substituent may be at least one kind selected from a group consisting of, for example, hydrogen, halogen, a cyano group, a nitro group, and a hydrocarbon group. The hydrocarbon group may be at least one kind selected from a group consisting of, for example, a C₁₋₄ alkyl group, a C₁₋₄ alkenyl group, and a C₁₋₄ alkynyl group.

The isothiocyanate may be chemically synthesized or extracted from a plant and purified. Examples of the plant include a cruciferous plant. Examples of the cruciferous plant include hon-wasabi (Wasabia japonica), horseradish (Armoracia rusticana), leaf mustard, cabbage, brussels sprouts, broccoli, cauliflower, bok choy, Brassica rapa, potherb mustard, turnip, kale, watercress, Japanese radish, thale cress, shepherd's purse, and maca. The 6-methylsulfinylhexyl isothiocyanate is particularly abundantly contained in hon-wasabi, particularly in an extract of the rootstock of hon-wasabi.

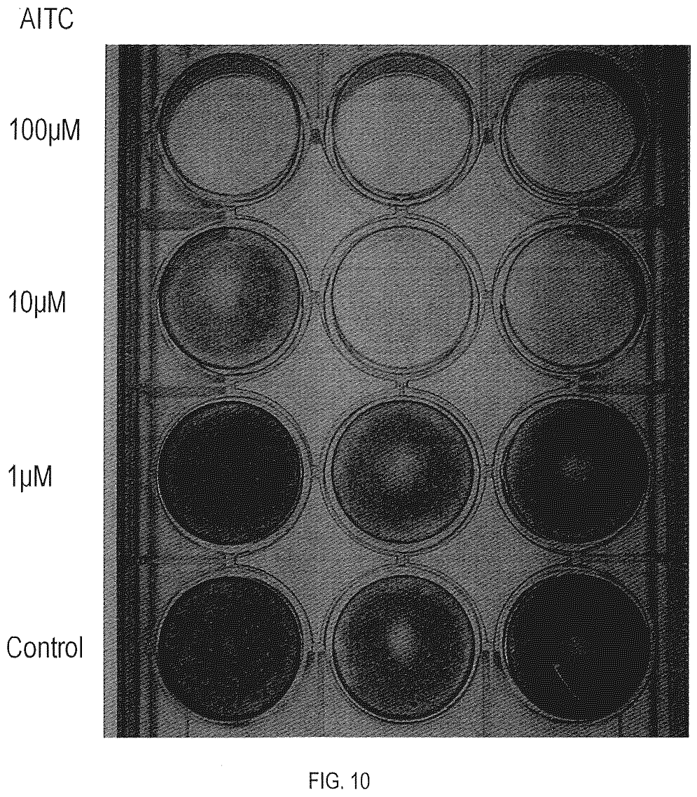

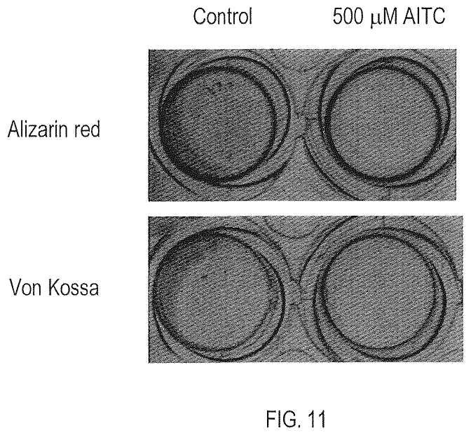

According to the study by the inventors of the present disclosure, some chemical compounds do not exhibit calcification promoting action even though they activate the TRPA1 channel. For example, an allyl isothiocyanate is reported to be a chemical compound that activates the TRPA1 channel. The 50% effective concentration (EC₅₀) of the allyl isothiocyanate is 64.53±3.12 M (Andrew H. et al. TRP channel activation by reversible covalent modification. Proc. Natl. Acad. Sci. USA. 2006 Dec. 19; 103 (51): 19564-19568.). However, according to the study by the inventors of the present disclosure, as shown in FIG. 10 and FIG. 11 showing the experimental results which will be explained later, the calcification action was not exhibited in a range up to 500 μM when the allyl isothiocyanate was added.

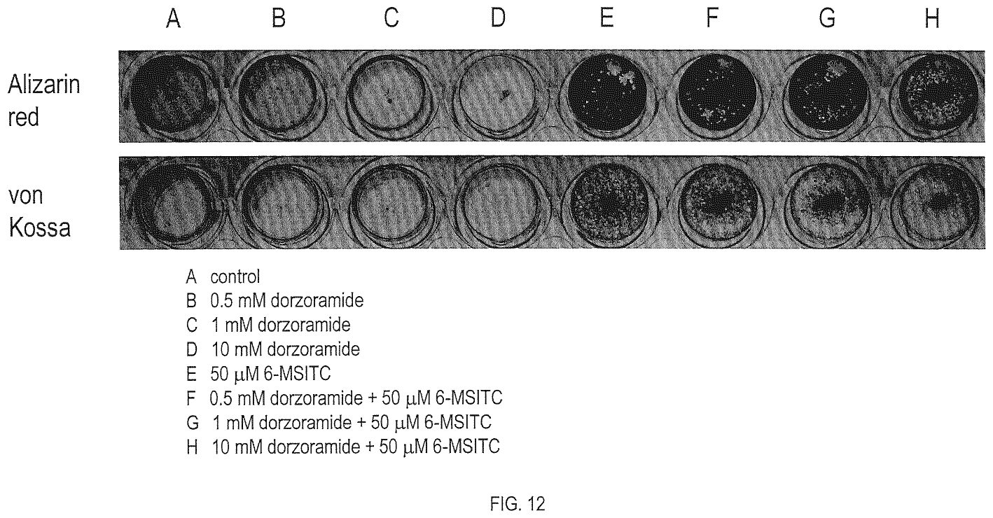

In addition, as shown in FIG. 12 showing the experimental result which will be explained later, it has been newly found that calcification is inhibited when dorzolamide, an inhibitor of the carbonic anhydrase, is used with the 6-methylsulfinylhexyl isothiocyanate. This findings has elucidated for the first time that not only the TRPA1 channel but also the carbonic anhydrase in the odontoblasts is involved in the calcification of the dentin. In other words, it has been suggested that the 6-methylsulfinylhexyl isothiocyanate is promoting the calcification of the dentin by not only activating the TRPA1 channel but also by activating a reaction catalyzed by the carbonic anhydrase in the odontoblasts.

Accordingly, it has been suggested that there is a possibility that a substance that promotes activation of the TRPA1 channel and activation of a reaction catalyzed by the carbonic anhydrase can promote calcification of the dentin. The following mechanisms of action can be considered as examples of the mechanisms of action by which the calcification is promoted by promotion of both of activation of the TRPA1 channel and activation of a reaction catalyzed by the carbonic anhydrase in the odontoblasts.
1) “TRPA1 and CA activation promoting composition” activates a TRP channel including the TRPA1 channel and other calcium ion inflow paths in the cell membranes as well as an intracellular calcium ion mobilization system and thereby increase an intracellular calcium ion concentration.
2) A calcium excretion system (sodium-calcium exchange/cell membrane Ca-ATPase, and an active transporter/passive transporter excreting calcium from cells, as well as vesicular transport) is activated and thus the calcium excretion from cells is promoted.
3) Meanwhile, “TRPA1 and CA activation promoting composition” activates an intracellular carbonic anhydrase. An intracellular bicarbonate ion generated by this activation is discharged from cells, which generates an alkaline environment outside cells.
4) Formation of the dentin is promoted by calcium deposition in the alkaline environment outside cells (at an area where the dentin is formed).

The calcification promoting action caused by the dental care composition and the “TRPA1 and CA activation promoting composition” of the present disclosure may be based on the aforementioned mechanism of action.

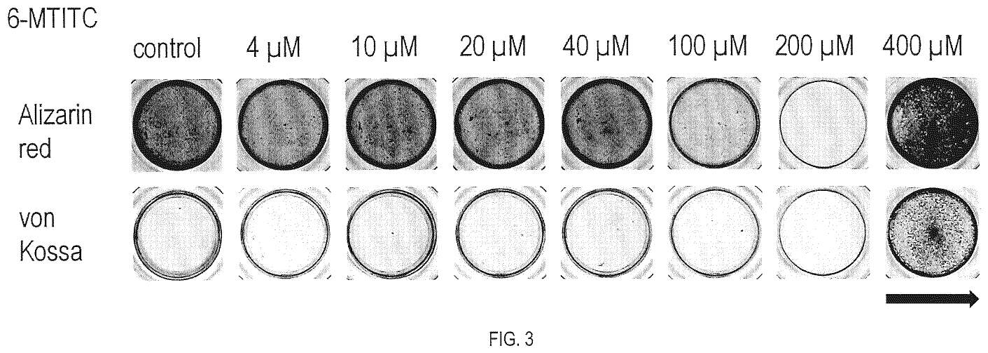

The aforementioned isothiocyanates include chemical compounds as to which it is not yet known whether the TRPA1 channel activation effect is exhibited. For example, it is not yet currently known whether the 8-methylsulfinyloctyl isothiocyanate, the 6-methyl sulphonyl isothiocyanate, and the like activate the TRPA1 channel; nevertheless, the study by the inventors of the present disclosure newly found that they have a calcification promoting action. It has been suggested that, similar to the 6-methylsulfinylhexyl isothiocyanate, those substances that are newly found this time as having the calcification promoting action, specifically, the 6-methylthiohexyl isothiocyanate, the 4-methylsulfinylbutyl isothiocyanate, the 8-methylsulfinyloctyl isothiocyanate, the 6-methylsulfonylhexyl isothiocyanate, and the phenethyl isothiocyanate shown in the experimental results in FIGS. 3 to 7, which will be described later, promote calcification of the dentin by activating the TRPA1 channel and a reaction catalyzed by the carbonic anhydrase in the odontoblasts.

Hereinafter, a composition containing an isothiocyanate will be given as one example of a composition that promotes calcification of a living body tissue, and the use of the composition, other components of the composition, and the method of use of the composition will be explained. Nevertheless, the composition that promotes calcification of a living body tissue is not limited to those that contain an isothiocyanate. Any compositions that are used to promote activation of the TRPA1 channel and a reaction catalyzed by the carbonic anhydrase can also be used in the use, contain other components of the composition, and be used with the method of use as described below.

[Use of Composition Containing Isothiocyanate]

As mentioned above, the isothiocyanate promotes formation, regeneration, and calcification of the dentin; and therefore, the isothiocyanate can be favorably used, for example, for dental care. The “dental care” means in this disclosure a therapeutic treatment and preventive treatment of illness and diseases that harm teeth or of related medical conditions, as well as strengthening, maintaining, and regenerating of the dental tissue. The illness and diseases that harm teeth or related medical conditions include, but are not limited to, exposure of the dentin caused by chipping of the enamel due to abrasion, dental caries, or the like; exposure of the dentin caused by regression of the gum due to periodontitis, aging, or the like; and hyperesthesia and the like caused by the exposure of the dentin. The isothiocyanate can be particularly used to strengthen the dental tissue, to prevent the hyperesthesia and/or improve the hyperesthesia, to promote formation, regeneration, and calcification of the dentin, and the like. In addition, as mentioned above, the isothiocyanate activates a reaction catalyzed by the carbonic anhydrase; therefore, the isothiocyanate can be used as a stimulating agent for the carbonic anhydrase in the odontoblast and as an activating agent for a reaction catalyzed by the carbonic anhydrase performed by the odontoblast and the like.

In other words, the isothiocyanate can be favorably used in applications for producing a dental care composition. Specifically, the isothiocyanate can be used in applications for producing a composition for strengthening the dental tissue, a composition to prevent and/or improve the hyperesthesia, a composition for promoting formation, regeneration, and calcification in the dentin, and the like. In addition, the isothiocyanate can also be used in applications to manufacture a composition for stimulating the carbonic anhydrase in the odontoblast and a composition to activate a reaction catalyzed by the carbonic anhydrase performed by the odontoblast, and the like.

The composition containing the isothiocyanate can be favorably used as, for example, food (particularly, food for dental care), a medicine and a quasi-drug (particularly, oral formulation), and an oral care product.

Examples of the food include a confectionery such as a chewing gum, a candy, a tablet-shaped sweet, a gummy candy, a chocolate, a snack, a rice cracker, a dry candy, a frozen confectionery, and other confectioneries such as a film-shaped confectionery and a soft capsule confectionery, dried food such as a dried squid, beverages, supplements, and other general processed food (including frozen food, etc.).

Examples of the chewing gum include a sheet-shaped gum, a sugar coated gum, and a bubble gum. Examples of the candy include hard candies (having the moisture content of less than 6% by mass) such as a drop, a taffy, and a butterscotch, and soft candies (having the moisture content of greater than or equal to 6% by mass) such as a caramel, a nougat, and a marshmallow. The tablet-shaped sweet is those that are formed into the shape of tablets. For example, it contains sugar as the main raw material which is optionally mixed with a binder, fruit juice, flavor, and the like, and is formed into the shape of a tablet. Examples of the tablet-shaped sweet include those called as lemonade flavored sweets and tablet sweets. The soft capsule confectionery is a confectionery formed by coating, for example, a liquid content or a paste content with a film. Examples of the beverages include a jelly beverage, a nutritional drink, a powder drink, and a soft drink. Examples of the supplements include those formed into a tablet, a granule, powder, and a soft capsule.

The composition containing the isothiocyanate can be favorably used particularly in food generally called a refreshing confectionery and food generally called functional food. The refreshing confectioneries are, for example, those that give a refreshing cool sensation in the oral cavity. Examples of the shape of the refreshing confectionery include the shapes of the aforementioned chewing gum, candy, tablet-shaped sweet, film-shaped confectionery, and soft capsule confectionery. The functional food means, for example, food that is approved to indicate its effects by a specified organization. Examples of the functional food include food for specified health uses, food with nutrient function claims, and food with functional claims.

Examples of the medicine and the quasi-drug include a lozenge, a buccal tablet, a chewable tablet, and a medicated gum.

Examples of the oral care product include a toothpaste, toothpowder, a dentifrice such as a liquid dentifrice, a mouthwash, a mouth rinse, a mouth spray, and an ointment for oral cavity.

[Other Components in Composition Containing Isothiocyanate]

The composition contains an effective amount of the isothiocyanate that is an active component. The “effective amount” means an amount of isothiocyanate that can achieve promotion of calcification in a living body tissue by an application onto the living body tissue. In addition to the isothiocyanate, various optional components that are generally used in food, medicine, quasi-drug, or oral care product may be optionally mixed to the composition as necessary to an extent that does not impair the effects of the present disclosure.

[Method for Using Composition Containing Isothiocyanate]

Formation, regeneration, and calcification of the dentin can be promoted by applying the composition containing the isothiocyanate to the teeth. In addition, strengthening of the dental tissues, and prevention and/or improvement of hyperesthesia can be achieved by applying the composition containing the isothiocyanate to the teeth.

When applying the composition containing the isothiocyanate to the teeth, it is preferable to have a liquid containing an isothiocyanate come into contact with the teeth in the oral cavity. For example, in a case where a composition is in a solid form, it is preferable that a part of the composition is soluble in saliva in the oral cavity such as the aforementioned chewing gum, candy, and lozenge. In this case, saliva (which is liquid) containing the isothiocyanate can come into contact with the teeth by taking the composition in the oral cavity. In a case where the composition containing the isothiocyanate is in a liquid form, the liquid containing the isothiocyanate can come into contact with the teeth by directly taking the composition in the oral cavity or by taking a liquid containing the diluted composition in the oral cavity.

The isothiocyanate is applied in the oral cavity such that the concentration of the isothiocyanate in the oral cavity (hereinafter, simply referred to as “intraoral concentration”) is greater than or equal to the concentration that is able to achieve promotion of calcification. For example, it is preferable to apply the composition in the oral cavity so that the intraoral concentration of the isothiocyanate is greater than or equal to 0.04 mol/m³ since a high calcification promoting action can be achieved. The intraoral concentration means the concentration of the isothiocyanate in the saliva when the composition containing the isothiocyanate is a solid form as mentioned above and at least a part of the composition is soluble in saliva in the oral cavity. In addition, the intraoral concentration means the concentration of the isothiocyanate in the composition when the composition containing the isothiocyanate is a liquid form; or the intraoral concentration means the concentration of the isothiocyanate in the diluted composition when the composition is diluted for an intake or a use.

Taking into consideration that the secretion volume, component, or the like of saliva vary between individuals, the following Formula 1 is used in the present disclosure to calculate the intraoral concentration of the isothiocyanate assuming a standard case when the composition containing the isothiocyanate is in a solid form.

$$[ Formula 1 ]$$

$$Intraoral Concentration [ mol / {m}^{3} ] = \frac{Amount of application of isothiocyanate per one use [ mol ]}{\begin{matrix}Average secretion volume of saliva [ {m}^{3} / min ] \times \\ Average stay time in oral cavity [ min ]\end{matrix}} \geq 0.04 \qquad ( 1 )$$

The “amount of application of isothiocyanate per one use” in Formula 1 is a total amount of isothiocyanate contained in the composition that is taken in the oral cavity in one intake when taking the composition containing the isothiocyanate in the oral cavity. Specifically, if there is a recommended intake or recommended amount per use of the composition containing the isothiocyanate, the amount of application of isothiocyanate per one use is the content of isothiocyanate in such recommended intake or recommended amount per use. For example, if the recommended intake or recommended amount per use is shown on a package of the composition containing the isothiocyanate or in an advertisement of the composition containing the isothiocyanate, the “amount of application of isothiocyanate per one use” is the content of isothiocyanate in such recommended intake or recommended amount per use. Meanwhile, if there is no particular recommended intake or recommended amount per use, the “amount of application of isothiocyanate per one use” is the content of isothiocyanate in a standard intake or in a standard amount of use of the composition containing the isothiocyanate.

Examples of cases where the recommended intake or recommended amount per use is shown on the package include, a case where a label on the package shows the recommended intake or recommended amount per use, a case where the description of the product accessible via a URL or a QR code (registered trademark) shown on a label on the packages show the recommended intake or recommended amount per use, and a case where the recommended intake or recommended amount per use is shown on descriptive pamphlet or the like attached to the package. The descriptive pamphlet attached to the package includes prescribing information sheet of a medicine or a quasi-drug. Examples of cases where the recommended intake or recommended amount per use is shown in the advertisement include, a case where the recommended intake or recommended amount per use is shown in a CM (Commercial Message), or in a website of the product. If there is a range in the recommended intake or recommended amount per use, the minimum value is adopted. For example, if it is recommended to take two to three products per one intake, the adopted recommended intake is two.

For example, in a case where the composition containing the isothiocyanate is in a product form that is individually separated, such as gums, candies or tablet-shaped sweets, if the recommended intake is two products, the “amount of application of isothiocyanate per one use” is the amount obtained by multiplying the content of isothiocyanate per one product by two. Meanwhile, if there are no particular recommended intake, the “amount of application of isothiocyanate per one use” is the content of isothiocyanate in one product since one is generally the minimum amount when taking a product.

For example, in a case where the composition containing the isothiocyanate is dentifrice or the like, if there is a recommended amount of use, the “amount of application of isothiocyanate per one use” is the content of isothiocyanate per the recommended amount of use. Meanwhile, if there are no particular recommended amount of use, the “amount of application of isothiocyanate per one use” is the content of isothiocyanate per 1 g of the product since the blending concentration of the medical component in a medical dentifrice in the category of quasi-drug is designed by assuming the use of 1 g of the product.

As the “average secretion volume of saliva” in Formula 1, 0.3 ml/min is adopted since the average secretion volume in the oral cavity at rest is about 0.3 ml/min (Shigeru Watanabe, “2. Knowledge of saliva for understanding oral infectious disease”, Oral Therapeutics and Pharmacology, Japanese Society of Oral Therapeutics and Pharmacology, 2016, Vol 35, third issue, p. 165-169).

The “average stay time in oral cavity” in Formula 1 is the average time the composition containing the isothiocyanate taken in the mouth stays in the oral cavity. For example, if the composition is in a product form that gradually dissolve in the oral cavity such as candies, the “average stay time in oral cavity” is an average time from the intake of the composition in the mouth to the dissolution to lose its shape. For example, if the composition is in a product form that is ultimately removed from the mouth such as gums, the “average stay time in oral cavity” is an average time from the intake to the removal of gums or the like from the mouth. The “average stay time in oral cavity” can be appropriately set depending on the size of the composition in a form of a product and on ingredient compositions. For example, assuming the general stay time in the oral cavity for each product form, the following time can be adopted as the “average stay time in oral cavity”.

For gums, it is generally about three to five minutes; therefore, three minutes is the “average stay time in oral cavity”.

For candies, it is generally about ten to fifteen minutes; therefore, ten minutes is the “average stay time in oral cavity”.

For tablet-shaped sweets, it is generally about twenty seconds to two minutes; therefore, twenty seconds is the “average stay time in oral cavity”.

For gummy candies, it is generally about ten to thirty seconds; therefore, ten seconds is the “average stay time in oral cavity”.

For chocolates, it is generally about ten to thirty seconds; therefore, ten seconds is the “average stay time in oral cavity”.

For lozenge, it is generally about seven to eight minutes; therefore, seven minutes is the “average stay time in oral cavity”.

Formula 1 is transformed into the following Formula 2.

[Formula 2]

Amount of application of isothiocyanate per one use [mol]=Intraoral Concentration [mol/m³]×Average secretion volume of saliva [m³/min]×Average stay time in oral cavity [min] (2)

The following Table 1 shows the amount of application of isothiocyanate per one use calculated based on Formula 2 and each of the aforementioned values when the intraoral concentration is greater than or equal to 0.04 mol/m³.

TABLE 1

| | Amount of |
|---|---|
| | Application |
| Form of Product | [μmol] |
| chewing gum | ≥0.036 |
| candy | ≥0.12 |
| tablet-shaped sweet | ≥0.012 |
| gummy candy | ≥0.0020 |
| lozenge | ≥0.084 |
| dentifrice | ≥0.036 |

The intraoral concentration is more preferably greater than or equal to 0.05 mol/m³, yet more preferably greater than or equal to 0.4 mol/m³, and particularly preferably greater than or equal to 0.5 mol/m³. The following Table 2 shows the amount of application of isothiocyanate per one use for each of these intraoral concentrations.

TABLE 2
Amount of Application [μmol]

| | Intraoral Concentration | | | |
|---|---|---|---|---|
| Form of | ≥0.04 | ≥0.05 | ≥0.4 | ≥0.5 |
| Product | mol/m³ | mol/m³ | mol/m³ | mol/m³ |
| chewing gum | ≥0.036 | ≥0.045 | ≥0.36 | ≥0.45 |
| candy | ≥0.12 | ≥0.15 | ≥1.2 | ≥1.5 |
| tablet-shaped | ≥0.012 | ≥0.015 | ≥0.12 | ≥0.15 |
| sweet | | | | |
| gummy candy | ≥0.0020 | ≥0.0025 | ≥0.02 | ≥0.025 |
| lozenge | ≥0.084 | ≥0.11 | ≥0.84 | ≥1.1 |
| dentifrice | ≥0.036 | ≥0.045 | ≥0.36 | ≥0.45 |

Although there are no upper limits on the intraoral concentration, the upper limit may be, for example, less than or equal to 5.0 mol/m³. The amounts of the application of the isothiocyanate per one use in this intraoral concentration is as shown in the following Table 3.

TABLE 3

| Amount of Application [μmol] | |
|---|---|
| | Intraoral Concentration |
| Form of Product | ≤5.0 mol/m³ |
| chewing gum | ≤4.5 |
| candy | ≤15 |
| tablet-shaped sweet | ≤1.5 |
| gummy candy | ≤0.25 |
| lozenge | ≤11 |
| dentifrice | ≤4.5 |

### EXAMPLES

Hereinafter, one mode of the present disclosure will be explained by providing examples. The present disclosure is nevertheless not limited to these examples.

### Example 1: Evaluation of Calcification Promoting Action Using Human Odontoblasts

1. Cultivation of Human Odontoblast (Human Odontoblast (HOB) Cells)

HOB cells are seeded in a T75 flask and cultivated until confluency was achieved. A culture medium (hereinafter referred to as “regular medium”) was prepared by adding 10% of fetal bovine serum (FBS) (Life Technologies Ltd.; Product No. 10437-028), 1% of penicillin-streptomycin (Life Technologies Ltd.; Product No. 15140-122), 1% of amphotericin B (Sigma-Aldrich, Product No. A2942-100ML) to MEMα (Life Technologies Ltd.; Product No. 12561-049, 12561-056). Cultivation was performed under an environment with a temperature of 37° C. and 5% of CO₂.

Then the HOB Cells were seeded on an adherent cell cultivation plate (Sumitomo Bakelite Co., Ltd) such that the cell density per 1 well was 1.78 to 2.56×10⁵ cells/mL, and cultivated under the same environment. As the adherent cell cultivation plate, plates having 6 to 96 wells were appropriately used.

Four days later, each culture medium was changed from the regular medium to a calcification medium, or to a culture medium (hereinafter, referred to as “additive containing calcification medium”) prepared by adding additional components shown in the following Table 4 and Table 5 to the calcification medium so as to achieve the concentrations shown in Table 4 and Table 5. The calcification medium is prepared by adding L (+)-ascorbic acid (Wako Pure Chemical Industries, Ltd. (currently Fujifilm Wako Pure Chemical Corporation), Product No. 016-04805) to the aforementioned regular mediums such that the concentration in the calcification medium (that is, the final concentration) was 50 μg/mL, and by adding β-glycerophosphoric acid (Fujifilm Wako Pure Chemical Corporation, Product No. 191-02042) to the aforementioned regular mediums such that the concentration in the calcification medium (that is, the final concentration) is 10 mM.

TABLE 4

| | | Concentration in additive |
|---|---|---|
| | | containing calcification |
| | Additional components | medium (i.e., final concentration) |
| Examples | 6-methylsulfinylhexyl isothiocyanate | 0.04 nM, 0.4 nM, 4 nM, 40 nM, |
| | (6-MSITC) (KINJIRUSHI Co., Ltd.) | 400 nM, 4 μM, 10 μM, 20 μM, |
| | | 40 μM, 100 μM, 200 μM, or |
| | | 400 μM, |
| | 6-methylthiohexyl isothio- | 4 μM, 10 μM, 20 μM, 40 μM, |
| | cyanate(6-MTITC) (KINJIRUSHI | 100 μM, 200 μM, or 400 μM, |
| | Co., Ltd.) | |
| | 6-methylsulfonylhexyl isothiocyanate | 0.5 nM, 5 nM, 50 nM, 5 μM, |
| | (6-MSFITC) (KINJIRUSHI Co., Ltd.) | 50 μM, or 100 μM, |
| | 4-methylsulfinylbutyl isothio- | 0.5 nM, 5 nM, 50 nM, 0.5 μM, |
| | cyanate(4-MSITC) (abcam plc; | 5 μM, 50 μM, or 100 μM, |
| | Product No. ab141969) | |
| | 8-methylsulfinyloctyl isothiocyanate | 0.5 nM, 5 nM, 50 nM, 0.5 μM, |
| | (8-MSITC) (abcam plc; Product No. | 5 μM, 50 μM, or 500 μM, |
| | ab142900) | |
| | phenethyl isothiocyanate (PITC) | 0.5 nM, 5 nM, 50 nM, 0.5 μM, |
| | (Sigma-Aldrich, Product No. 253731-5G) | 5 μM, 50 μM, or 500 μM, |
| Comparative | 4-methylsulfinylbutyl amine (4-MS | 0.5 nM, 5 nM, 50 nM, 5 μM, |
| Examples | amine) (Santa Cruz Biotechnology, | 50 μM, or 500 μM, |
| | Product No. sc-216711) | |
| | n-hexyl isothiocyanate (n-Hexyl ITC) | 0.5 nM, 5 nM, 50 nM, 0.5 μM, |
| | (Sigma-Aldrich, Product No. 475912-5G) | 5 μM, 50 μM, or 500 μM, |
| | allyl isothiocyanate (AITC) (Wako Pure | 1 μM, 10 μM, 100 μM, or |
| | Chemical Industries, Ltd. (currently | 500 μM, |
| | Fujifilm Wako Pure Chemical | |
| | Corporation), Product No. 016-01463) | |

TABLE 5

| | | Concentration in additional |
|---|---|---|
| | | components and additive |
| | | containing calcification medium |
| | | (i.e., final concentration) |
| Reference | A | — |
| Examples | B | 0.5 mM dorzolamide (Sigma Aldrich, |
| | | Product No. 1225281) |
| | C | 1 mM dorzolamide |
| | D | 10 mM dorzolamide |
| | E | 50 μM 6-MSITC |
| | F | 0.5 mM dorzolamide and 50 μM 6-MSITC |
| | G | 1 mM dorzolamide and 50 μM 6-MSITC |
| | H | 10 mM dorzolamide and 50 μM 6-MSITC |

Then, the cultivation was performed under the environment at 37° C. with 5% of CO₂ for 21 or 28 days while exchanging the calcification medium or the additive containing calcification medium twice a week. Then the following staining was performed.

2. Staining

Alizarin red staining and von Kossa staining were performed to evaluate calcification promoting effect. Only the Alizarin red staining was performed for the cases where 1 μM, 10 μM, and 100 μM of the allyl isothiocyanate were added.

(1) Alizarin Red Staining

Each well was washed twice using Dulbecco's phosphate buffered saline (DPBS) (Life Technologies Ltd.; Product No. 14190-144) the amount of which was equal to the specified amount of culture medium in the adherent cell cultivation plate. After removing the DPBS from each well, 4% paraformaldehyde phosphate buffer solution (Fujifilm Wako Pure Chemical Corporation, Product No. 163-20145), the amount of which was equal to a half the amount of the specified amount of the culture medium, was introduced to each well, and each well was left for five minutes at a room temperature.

Then, each well was washed twice using the DPBS the amount of which was equal to the specified amount of the culture medium. After removing the DPBS from each well, Alizarin red staining solution, the amount of which was equal to a half the amount of the specified amount of the culture medium, was introduced to each well, and each well was left for ten minutes. The Alizarin red staining solution was prepared by dissolving a commercially available staining solution (Product name: “Alizarin red S”, Wako Pure Chemical Industries, Ltd. (currently Fujifilm Wako Pure Chemical Corporation), Product No. 011-01192) with ultrapure water (Milli-Q (registered trademark) water), and the pH was adjusted to be within a range from 6.36 to 6.40 using ammonium hydroxide solution.

Each well was then washed five times using the ultrapure water (same as above), the amount of which was equal to the specified amount of the culture medium.

(2) Von Kossa Staining

Each well was washed three times using the Dulbecco's phosphate buffered saline (DPBS) (same as above) the amount of which was equal to the specified amount of the culture medium in the adherent cell cultivation plate. After removing the DPBS from each well, 4% paraformaldehyde phosphate buffer solution (same as above), the amount of which was equal to a half the amount of the specified amount of the culture medium, was introduced to each well, and each well was left for ten minutes at a room temperature.

Then, each well was washed twice using the ultrapure water (same as above), the amount of which was equal to the specified amount of the culture medium. After removing the ultrapure water from each well, 5% AgNO₃ aqueous solution (AgNO₃ is a product of Fujifilm Wako Pure Chemical Corporation, Product No. 194-00832), the amount of which was equal to the specified amount of the culture medium, was introduced to each well, and each well was left for one hour under a fluorescent light.

Each well was then washed twice using the ultrapure water (same as above), the amount of which was equal to the specified amount of the culture medium. After removing the ultrapure water from each well, 0.5% sodium thiosulfate aqueous solution (sodium thiosulfate is a product of Wako Pure Chemical Industries, Ltd. (currently Fujifilm Wako Pure Chemical Corporation), Product No. 197-03605), the amount of which was equal to the specified amount of the culture medium, was introduced to each well, and each well was left for one to two minutes at a room temperature.

Each well was then washed three times using the ultrapure water (same as above), the amount of which was equal to the specified amount of the culture medium.

(3) Analysis

The HOB cells after the staining were photographed per each well using a digital camera (digital single-lens reflex camera α6300, Sony Corporation), the photographed images were analyzed by using an image processing software Image J (developed by NIH).

3. Discussion

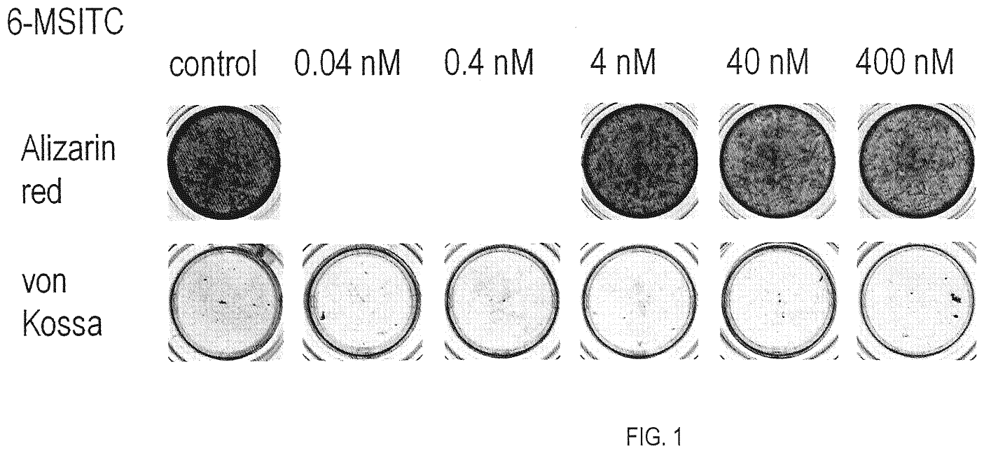

The results of the analysis are shown in FIG. 1 to FIG. 12.

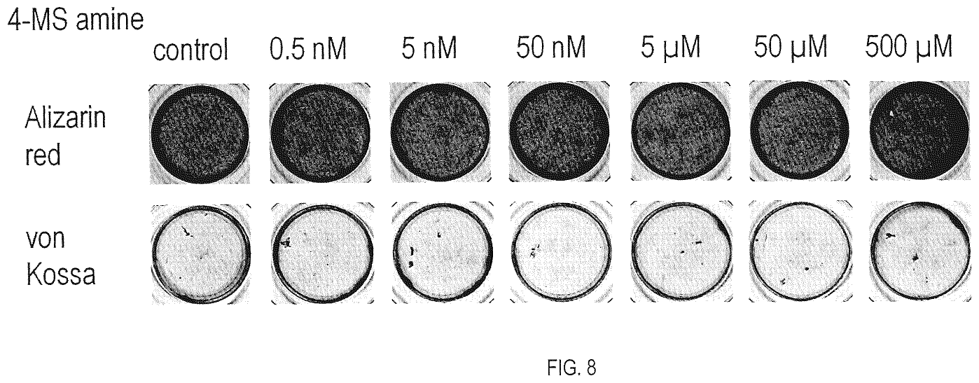

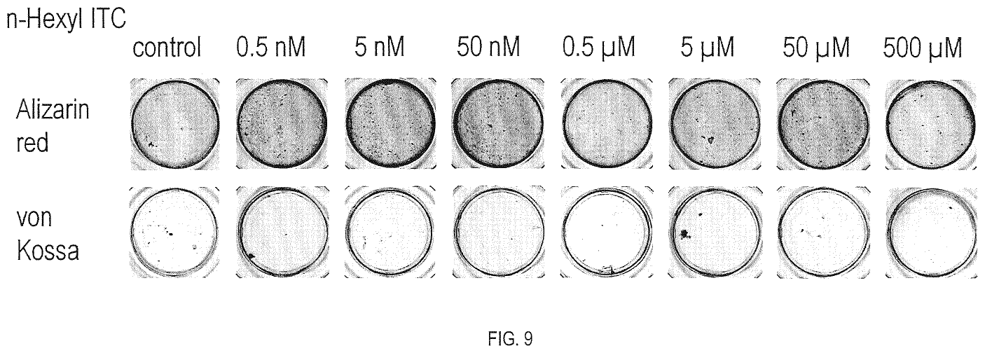

As shown in FIG. 1 to FIG. 7, it was found that the 6-methylsulfinylhexyl isothiocyanate (6-MSITC) having a sulfinyl group; the 6-methylthiohexyl isothiocyanate (6-MTITC), the 4-methylsulfinylbutyl isothiocyanate (4-MSITC), and the 8-methylsulfinyloctyl isothiocyanate (8-MSITC) having a sulfide bond; the 6-methylsulfonylhexyl isothiocyanate (6-MSFITC) having a sulphonyl group; and the phenethyl isothiocyanate (PITC) having an aryl group possess calcification promoting action. Meanwhile, as shown in FIG. 8, the 4-methylsulfinylbutyl amine (4-MS amine), which is not an isothiocyanate, showed completely no calcification promoting action. As shown in FIG. 9, the n-hexyl isothiocyanate (n-Hexyl ITC) having no substituent showed completely no calcification promoting action. As shown in FIG. 10 and FIG. 11, the allyl isothiocyanate (AITC) having an allyl group showed completely no calcification promoting action.

According to these results, is has been suggested that an isothiocyanate group (—N═C═S) may play an important part in the calcification promoting action. It has also been suggested that it may be important in the calcification promoting action that an isothiocyanate has a substituent. In addition, it has also been suggested that a sulfinyl group (—S(═O)—), a sulphonyl group (—S(═O)₂—), a sulfide bond (—S—), and an aryl group are substituents that play important parts in the calcification promoting action.

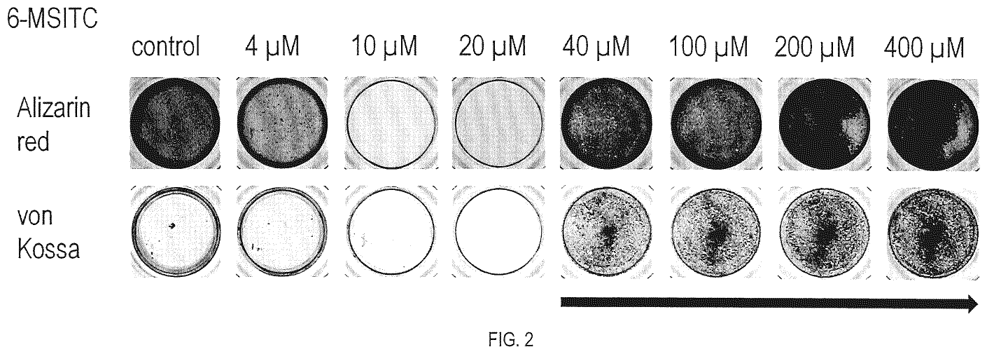

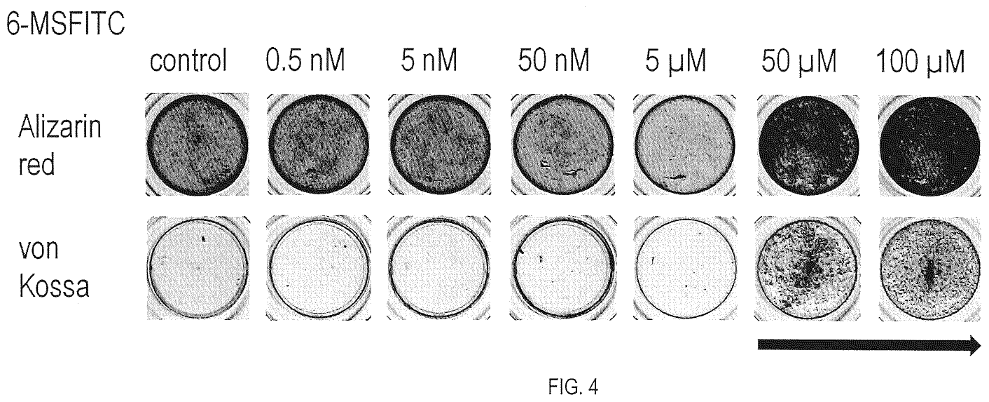

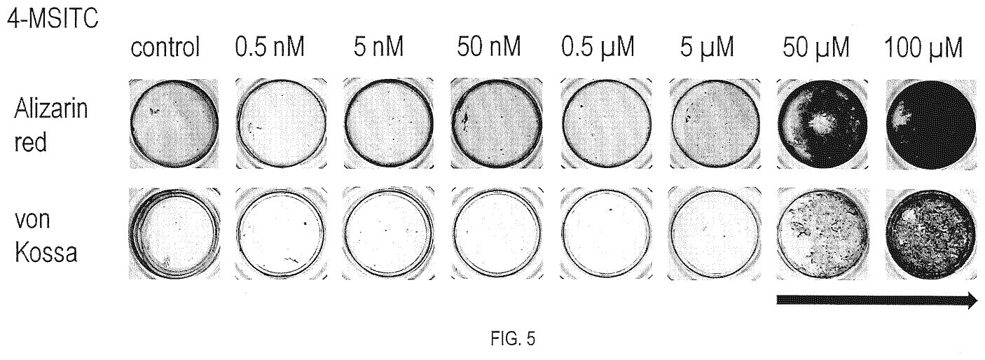

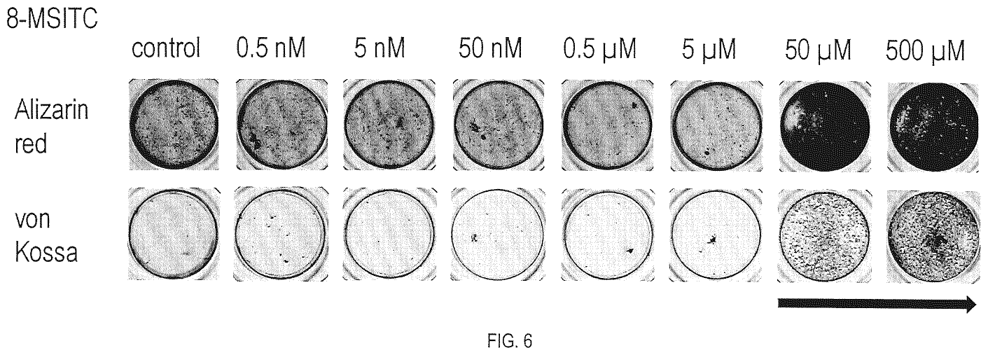

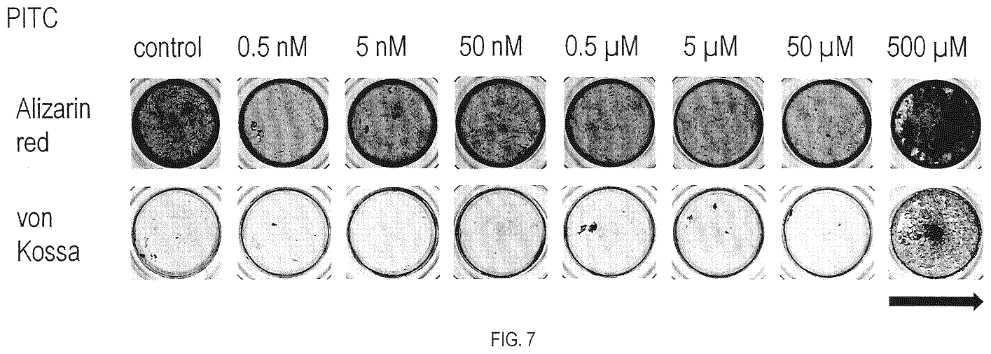

As shown in FIG. 1 to FIG. 2, particularly high calcification promoting action was observed at 40 μM (=0.04 mol/m³) or greater regarding the 6-MSITC. As shown in FIG. 3, particularly high calcification promoting action was observed at 400 μM (=0.4 mol/m³) or greater regarding the 6-MTITC. As shown in FIG. 4, particularly high calcification promoting action was observed at 50 μM (=0.05 mol/m³) or greater regarding the 6-MSFITC. As shown in FIG. 5, particularly high calcification promoting action was observed at 50 μM (=0.05 mol/m³) or greater regarding the 4-MSITC. As shown in FIG. 6, particularly high calcification promoting action was observed at 50 μM (=0.05 mol/m³) or greater regarding the 8-MSITC. As shown in FIG. 7, particularly high calcification promoting action was observed at 500 μM (=0.05 mol/m³) or greater regarding the PITC.

According to these results, the calcification promoting action was particularly high in the 6-MSITC, 6-MSFITC, 4-MSITC, and 8-MSITC, among which, the calcification promoting action of 6-MSITC was the highest. This has suggested that the isothiocyanate, whose carbon number (excluding the carbon number of the substituent at the terminal) of the alkyl group having a substituent at its terminal is 4 to 8, is preferable; and that the isothiocyanate, whose carbon number (excluding the carbon number of the substituent at the terminal) of the alkyl group having a substituent at its terminal is 6, is more preferable.

Regarding the 6-MSFITC and the 4-MSITC, since deposits of calcium is greater (that is, the extent of calcification is greater) in the 6-MSFITC than in the 4-MSITC comparing the extent of staining at 50 μM, it has been suggested that the 6-MSFITC has higher calcification promoting action than the 4-MSITC. Therefore, it has been suggested that the calcification promoting action is higher in the order of the 6-MSITC>the 6-MSFITC>the 4-MSITC. The calcification promoting action of the 8-MSITC was about the same as the calcification promoting action of the 4-MSITC.

By comparing the concentrations where the aforementioned particularly high calcification promoting action was observed in the 6-MSITC, the 6-MTITC, the 6-MSFITC, and the PITC, it has been suggested that, as the substituent of the isothiocyanate, contribution to the calcification promoting action is greater in the order of the sulfinyl group (—S(═O)—)>the sulphonyl group (—S(═O)2-)>the sulfide bond (—S—)>the aryl group.

As shown in FIG. 12, calcification promoting action was not observed in a case where the 6-MSITC and dorzolamide, an inhibitor of the carbonic anhydrase, were added in combination. This has suggested that the 6-methylsulfinylhexyl isothiocyanate may promote calcification in the dentin not only by activating the TRPA1 channel, but also by activating the carbonic anhydrase. It has also been suggested that, in the odontoblast, the substance that is able to activate the TRPA1 channel and a reaction catalyzed by the carbonic anhydrase may be able to promote calcification in the dentin.

### Example 2: Analysis of Reactionary Dentine Forming Ability of 6-methylsulfinylhexyl isothiocyanate (6-MSITC) In Vivo

1. Analysis of Reactionary Dentine Forming Ability of 6-MSITC Using Rat

An animal used in the analysis (Wistar rat as mentioned below) was in a condition not having any specific pathogens and raised housed in a transparent cage, where woodchips were spread, under a light and dark cycle (12 hour: 12 hour) (temperature: 21 to 23° C.; humidity: 40 to 60%) with free access to food and water. The animal was treated in accordance with “Guiding principles for the care and use of animals in the field of physiological science” provided by the Physiological Society of Japan and the American Physiological Society. The experiments were carried out in accordance with the Guideline for the Care and Use of Laboratory Animals” provided by the National Institutes of Health in the United States. All the experiments in this study were carried out under the approval of the Institutional Animal Care and Use Committee of the Tokyo Dental College (Animal Experiment Plan Approval No. 190301, 200301). Every possible effort was made to minimize the pain for the animals. The animals used in this study were healthy and no complications were observed during the period of experiment.

A Wistar rat was anesthetized using 3% of isoflurane (Fujifilm Wako Pure Chemical Corporation). A cavity was formed in each of the right and left lower first molars of the same rat; the cavities were formed from the enamel to the dentin to expose the dentin. The cavities were formed by using a dental motor (VIVA MATE G5, Nakanishi Inc.), a low-speed handpiece (Torgtech, J. Morita Co.), and a dental diamond bar (Shofu Inc.). A MedGel (P15 sheet) (Nitta Gelatin Inc.) immersed in saline solution was placed on the exposed surface of the dentin of the left lower first molar, and the exposed dentin was blocked by a dental lining material (Ionosit baseliner, DMG, Germany) (a group administrated with saline solution; control). A MedGel (P15 sheet) (same as above) immersed in 500 μM of the 6-MSITC was placed on the exposed surface of the dentin of the right lower first molar, and the exposed dentin was blocked by a dental lining material (same as above) (a group administered with 500 μM of the 6-MSITC).

Two weeks after administration, the rat was euthanized with 3% of isoflurane (same as above) and three types of mixed anesthetic (domitor, midazolam, and vetorphale) to obtain samples of both mandibles. The obtained sample was scanned by using a micro CT (μCT-50, Scano Medical, Switzerland). The scanning conditions were as follows: the tube voltage was 70 kV, the tube current was 200 μA, and the image acquisition resolutions were x=10 m, y=10 m, and z=10 m. The extent of the dentin formation was evaluated by using thus obtained sliced images. In addition, using an image analyzing software (TRI/3D-BON, RATOC System Engineering Co.) and a volume rendering method, a three-dimensional construction image was obtained by three-dimensional structuring the sliced data; the obtained three-dimensional construction image was used to perform a morphological observation.

2. Discussion

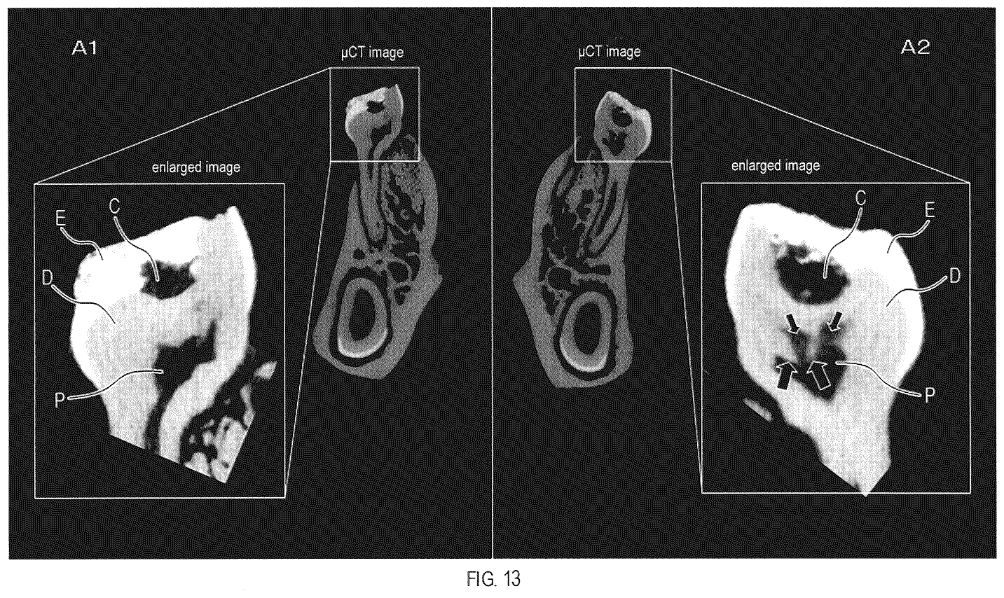

As pointed by thick arrows and narrow arrows in the enlarged images of the micro CT (μCT) images shown in FIG. 13, apparently new reactionary dentine formation was observed in the group administered with 500 μM of the 6-MSITC (A2) compared with the group administrated with saline solution (control; A1). Judging from the radiolucency, it was observed that the reactionary dentine includes low calcification areas (areas pointed by the narrow arrows) and high calcification areas (areas pointed by the thick arrows).

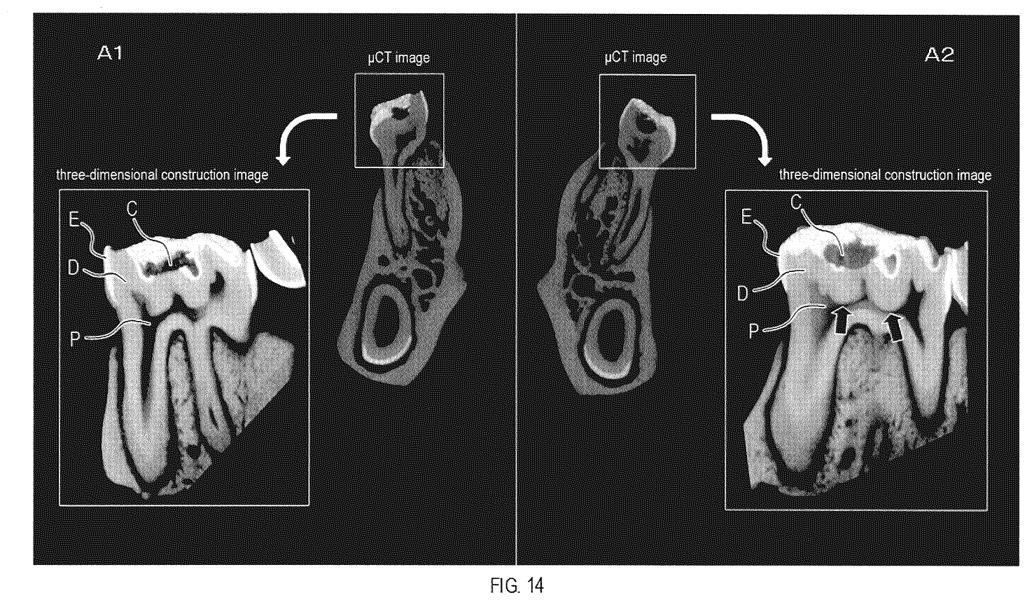

As pointed by thick arrows in the three-dimensional construction image shown in FIG. 14, a reactionary dentine formation, so large that it came close to the floor of the pulp chamber, was observed in the group administered with 500 μM of the 6-MSITC.

As mentioned above, promotion of calcification by the 6-MSITC was also confirmed in Vivo. Accordingly, it was shown that, calcification of the dentin of the teeth can actually be promoted by having the 6-MSITC come into contact with the teeth. In this experiment, calcification occurred in only two weeks after having the 6-MSITC come into contact with the teeth. It was therefore found that the period of time until the effect can be seen is remarkably short. For example, in a case where calcium hydroxide or MTA (Mineral Trioxide Aggregate) generally used in dental clinical practices is used in human, the normal duration of time until calcification occurs is three months. Compared to this, the duration of time until calcification by the 6-MSITC occurs is remarkably short as mentioned above. This leads to an expectation that the 6-MSITC makes it possible to render the calcification effect in a shorter period of time than the conventional medical agent does. Accordingly, it has been suggested that the dental care composition, and the TRPA1 and CA activation promoting composition of the present disclosure can be favorably used to strengthen the dental tissue, prevent the hyperesthesia and/or improve the hyperesthesia, promote calcification of the dentin, and the like.

1. A dental care composition comprising:
an isothiocyanate having a sulfinyl group (—S(═O)—), a sulphonyl group (—S(═O)₂—), a sulfide bond (—S—), or an aryl group, and an isothiocyanate group (—N═C═S).

2. The dental care composition according to claim 1, wherein the isothiocyanate include a methylsulfinylalkyl isothiocyanate.

3. The dental care composition according to claim 1, wherein the isothiocyanate includes a 6-methylsulfinylhexyl isothiocyanate.

4. The dental care composition according to claim 3,
wherein the dental care composition is a chewing gum,
wherein, if the dental care composition has a recommended intake per use, a content of the isothiocyanate in the recommended intake per use may be greater than or equal to 0.036 μmol, or
wherein, if the dental care composition does not have a recommended intake per use, the content of the isothiocyanate in one piece of the dental care composition is greater than or equal to 0.036 μmol.

5. The dental care composition according to claim 3,
wherein the dental care composition is a candy,
wherein, if the dental care composition has a recommended intake per use, the content of the isothiocyanate in the recommended intake per use is greater than or equal to 0.12 μmol, or
wherein, if the dental care composition does not have a recommended intake per use, the content of the isothiocyanate in one piece of the dental care composition is greater than or equal to 0.12 μmol.

6. The dental care composition according to claim 3,
wherein the dental care composition is a tablet-shaped sweet,
wherein, if the dental care composition has a recommended intake per use, the content of the isothiocyanate in the recommended intake per use is greater than or equal to 0.012 μmol, or
wherein, if the dental care composition does not have a recommended intake per use, the content of the isothiocyanate in one piece of the dental care composition is greater than or equal to 0.012 μmol.

7. The dental care composition according to claim 3,
wherein the dental care composition is a lozenge,
wherein, if the dental care composition has a recommended intake per use, the content of the isothiocyanate in the recommended intake per use is greater than or equal to 0.084 μmol, or
wherein, if the dental care composition does not have a recommended intake per use, the content of the isothiocyanate in one piece of the dental care composition may be greater than or equal to 0.084 μmol.

8. The dental care composition according to claim 3,
wherein the dental care composition is a dentifrice,
wherein, if the dental care composition has a recommended amount per use, the content of the isothiocyanate in the recommended amount per use is greater than or equal to 0.036 μmol, or
wherein, if the dental care composition does not have a recommended amount per use, the content of the isothiocyanate in 1 g of the dental care composition is greater than or equal to 0.036 μmol.

9. The dental care composition according to claim 1, wherein the dental care composition is used for one or more purposes selected from improvement of hyperesthesia and prevention of hyperesthesia.

10. The dental care composition according to claim 1, wherein the dental care composition is used to strengthen dental tissues.

11. The dental care composition according to claim 1, wherein the dental care composition is used to promote calcification of dentin.

12. A method of promoting calcification of dentin (excluding medical practices to a human), the method comprising:
applying a dental care composition to a tooth, the dental care composition including an isothiocyanate having a sulfinyl group (—S(═O)—), a sulphonyl group (—S(═O)₂—), a sulfide bond (—S—), or an aryl group, and an isothiocyanate group (—N═C═S).

13. The method of promoting calcification of the dentin according to claim 12,
wherein the isothiocyanate includes a methylsulfinylalkyl isothiocyanate.

14. The method of promoting calcification of the dentin according to claim 12,
wherein the isothiocyanate includes a 6-methylsulfinylhexyl isothiocyanate.

15. The method of promoting calcification of the dentin according to claim 12,
wherein the dental care composition is a composition, at least a part of which dissolves in saliva in an oral cavity, and
wherein the method includes applying the dental care composition into an oral cavity such that the concentration of the isothiocyanate in the saliva is greater than or equal to 0.04 mol/m³.

16. A method of promoting calcification of dentin (excluding medical practices to a human), the method comprising:
having a liquid containing an isothiocyanate come into contact with a tooth, the isothiocyanate having a sulfinyl group (—S(═O)—), a sulphonyl group (—S(═O)₂—), a sulfide bond (—S—) or a phenyl group, and an isothiocyanate group (—N═C═S),
wherein a concentration of the isothiocyanate in the liquid is greater than or equal to 0.04 mol/m³.

17. A composition used to promote activation of a TRPA1 channel and to promote activation of a reaction catalyzed by carbonic anhydrase.

18. The composition according to claim 17 further comprising:
an isothiocyanate (excluding an allyl isothiocyanate and an n-alkyl isothiocyanate) as an active component.

19. The composition according to claim 18,
wherein the isothiocyanate is at least one of a 6-methylsulfinylhexyl isothiocyanate, a 6-methylthiohexyl isothiocyanate, or a 6-methylsulfonylhexyl isothiocyanate.
